(19) United States
(12) Patent Application Publication
(10) Pub. No.: US 2004/0023994 A1
(43) Pub. Date: Feb. 5, 2004

(54) Glutathione-activated anti-tumor prodrugs of 6-mercaptopurine and 6-thioguanine

(76) Inventors: Adnan A. Elfarra, Madison, WI (US); Sjofn Gunnarsdottir, Madison, WI (US)

Correspondence Address: Bennett J. Berson, Quarles & Brady LLP, P O Box 2113, Madison, WI, 53701-2113, US

(21) Appl. No.: 10/454,443

(22) Filed: Jun. 4, 2003

Related U.S. Application Data
(60) Provisional application No. 60/386,504, filed on Jun. 4, 2002.

(51) Int. Cl. A61K 31/522

(52) U.S. Cl. 514/263.3; 514/263.38

(57) ABSTRACT

The present invention relates to a method for treating a tumor in a tissue of a human or non-human animal, the tumor having an elevated level of glutathione relative to the tissue, the method comprising the steps of administering to the animal a prodrug comprising a thiopurine having a sulfur heteroatom conjugated to an alpha-, beta-unsaturated carbonyl moiety, in combination with a pharmaceutically acceptable carrier, the moiety comprising a double bond having an alpha end and a beta end, the beta end being accessible to glutathione in an addition-elimination reaction, the prodrug lacking an ionizable carboxylic acid group; and observing a reduction in growth of the tumor.

### CROSS-REFERENCE TO RELATED APPLICATIONS

This application claims the benefit of U.S. Provisional Application No. 60/386,504 filed Jun. 4, 2002 which is incorporated herein by reference in its entirety.

### STATEMENT REGARDING FEDERALLY SPONSORED RESEARCH OR DEVELOPMENT

This invention was made with United States government support awarded by the following agencies: NIH DK 44295. The United States has certain rights in this invention.

### BACKGROUND OF THE INVENTION

When administered in vivo, cytotoxic antimetabolites 6-mercaptopurine (6-MP) and its analog 6-thioguanine (6-TG) form nucleosides and nucleotides that can interfere with multiple cellular processes including incorporation into DNA and/or RNA. Absorption after oral administration of the thiopurines is incomplete and variable, with up to 46% and 37% of the dose absorbed after 6-TG and 6-MP administration, respectively. The plasma half-life of the thiopurines is relatively short, while the half-life of nucleotides in tissues is longer. 6-TG is more potent cytotoxic agent than 6-MP, but is also more toxic to the recipient. The primary and most severe adverse effect associated with 6-TG and 6-MP use is bone marrow toxicity with leukopenia being most commonly observed followed by thrombocytopenia and anemia. Gastrointestinal and hepatic side effects have also been observed.

Since the 1950s, these thiopurines have been used to treat leukemias. Even though 6-MP and 6-TG are most commonly used to treat leukemias, both possess growth inhibitory and cytotoxic activity against several solid tumor cell lines in vitro. However, clinical use of thiopurine drugs against solid tumors has been hampered because bone marrow toxicity and, to a lesser extent, gastrointestinal toxicity are dose limiting factors (Frank and Tornyos, 1962; Regelson et al., 1964; Moore et al., 1968). Therefore, by developing prodrugs that improve the targeting of the thiopurines to the tumor cells, it may be possible to decrease systemic toxicity and thereby increase clinical use of these agents.

It is of considerable interest to note that many tumors have abnormal thiol metabolism and changes in the expression of many thiol metabolizing enzymes as well as in thiol levels have been well characterized. Moreover, increased glutathione (GSH) levels have been linked with chemotherapeutic drug resistance. A high level of glutathione is considered to be at least in the range of 1-10 mM in humans, although this number varies greatly from species to species, depending upon tissue- and cell type.

GSH is important for many cellular processes and is crucial for detoxifying and excreting many chemotherapeutic drugs from cells. Cellular thiols such as GSH and thioredoxin are important in regulating intracellular oxidation-reduction (redox) status which is itself important in regulating apoptosis. Because many tumors exhibit abnormal cellular thiol metabolism in vivo and have increased levels of glutathione (GSH) relative to surrounding normal tissue, an effort was undertaken to develop prodrugs that are metabolized more rapidly, or in greater quantity, in such tumors than in surrounding tissue. Such prodrugs may not only selectively deliver a chemotherapeutic moiety to the tumor cell, but may also perturb the cellular thiol homeostasis and thereby enhance the effects of the chemotherapeutic drug on the tumor cell. It was postulated that related prodrugs having therapeutic efficacy relatively higher than the parent drug could be engineered such that it would be possible to administer the prodrugs to avoid the toxic side effects of the parent drugs.

Gunnarsdottir, S. and A. A. Elfarra, “Glutathione-Dependent Metabolism of cis-3-(9H-Purin-6-ylthio)acrylic Acid to Yield the Chemotherapeutic Drug—Mercaptopurine: Evidence for Two Distinct Mechanisms in Rats,” J. Pharmacol. Exp. Ther. 290: 950 (1999), incorporated herein by reference as if set forth in its entirety, demonstrated that cis-3-(9H-purin-6-ylthio)acrylic acid (PTA), a propenoic acid conjugate prodrug of 6-MP, yields 6-MP via two distinct GSH-dependent pathways. First, 6-MP is formed directly from PTA, a Michael acceptor, via an addition-elimination reaction with GSH. Second, S-(9H-purine-6-yl) glutathione (PG), the major metabolite formed in the reaction between PTA and GSH, is further metabolized to 6-MP.

Due to the slow reactivity of PTA towards GSH, attention shifted to a class of alpha, beta-unsaturated PTA analogs having a sulfur heteroatom conjugated to a butenone moiety. This class of analogs was expected to react more efficiently with GSH than PTA to yield 6-MP and 6-TG, because these analogs lack an ionizable carboxylic acid group that was believed to decrease the reactivity of PTA towards GSH. Two compounds of interest have been studied, namely 6-(2-acetyl vinyl thio) guanine (AVTG) and 6-(2-acetyl vinyl thio) purine (AVTP). These compounds were described by Anufiiev, M. A. et al., 2-Acylvinylation of sulfur-containing pyrines, J. Gen. Chem. USSR 52:884 (1982), incorporated herein by reference as if set forth in its entirety, but no characterization of biological activity was reported cis-AVTP and trans-AVTG have been studied more extensively than other geometrical isomers, as these isomers are present in higher amount after synthesis.

In cell culture, AVTG uptake and metabolic conversion to 6-TG was associated with a concomitant decrease in cellular GSH levels, with less intracellular 6-TG being obtained in GSH-depleted cells than in cells having intact GSH levels. Moreover, incubation with AVTG resulted in higher intracellular 6-TG levels than treatment with an equimolar concentration of 6-TG. Thus, the prodrug delivered more 6-TG to the cell than did 6-TG itself. AVTG and AVTP had similar IC₅₀ values that are comparable to those of 6-TG, but significantly lower than those of 6-MP.

Also, AVTP exhibited dose-dependent increase in cytotoxicity in human renal cell carcinoma cell lines ACHN and A-498, whereas PTA caused little cytotoxicity, even at concentrations up to 500 μM. Cytotoxicity was similar or higher after treatment with AVTP than after treatment with 6-MP or with its analog azathioprine.

Various thiopurine drugs, prodrugs and analogs are shown in FIG. 1.

### BRIEF SUMMARY OF THE INVENTION

The present invention is summarized in that a method for treating in vivo a tumor having an increased level of glutathione relative to surrounding normal tissue includes the step of administering to a human or non-human animal a prodrug of a thiopurine, the prodrug having a sulfur heteroatom conjugated to an alpha-, beta-unsaturated carbonyl moiety and lacking an ionizable carboxylic acid group, according to a delivery regimen sufficient to reduce or prevent tumor growth, for example by reducing the diameter of an existing solid tumor by at least 10 percent. The methods of the invention are equally well suited for treating blood-borne tumors, where reduction or prevention of growth can be characterized using conventional methods.

The invention is also summarized in that the alpha-, beta-unsaturated carbonyl moiety comprises a double bond having an alpha end and a beta end, the beta end being accessible to glutathione in an addition-elimination reaction. The applicants appreciate the importance of maintaining in the prodrugs an attack site for GSH at the beta end of the double bond (i.e., closer to the sulfur atom). Along these lines, it is understood by the applicants that the presence in the alpha, beta-unsaturated carbonyl moiety of at least 3 carbons, and more preferably four carbons, having a GSH-accessible double bond and the absence of an ionizable carboxylic acid group have advantageous benefit.

In another related embodiment, the carbonyl moiety can comprise an amine group attached to the terminal carbon.

In yet another related embodiment, the thiopurine is selected from the group consisting of trans-AVTG and cis-AVTP.

The skilled artisan will further appreciate that the carbon at the beta end of the alpha, beta-unsaturated carbonyl group should remain connected to the sulfur atom. In contrast, the position adjacent to the carbonyl group can alternatively be a hydrogen, an amine, an aryl group or an alkyl chain (branched or unbranched) having 10 or fewer carbons. Alkyl chains and aryl groups at that position can also include functional groups such as hydroxyl groups, amines or the like to maintain or increase the solubility of the prodrug in the carrier. Still further, any hydrogen atom in the double bond itself can be replaced with a substituted or unsubstituted alkyl chain (branched or unbranched) or aryl group. It is also appreciated that to further enhance the activity of the ultimate drug, changes to the purine portion of the prodrug can be made without departing from the spirit of the invention. Applicants thereby define a broader class of suitable prodrugs within the scope of the invention.

The invention is further summarized in that a prodrug of the invention is combined with a pharmaceutically acceptable carrier before administration.

It is an object of the present invention to treat a tumor that is up-regulated for GSH.

It is another object of the present invention to treat a tumor with a prodrug of a thiopurine drug that is more cytotoxic for tumor cells than the thiopurine drug.

It is a feature of the present invention that AVTG and AVTP are comparably or more cytotoxic to target tumor cells than the corresponding thiopurine drugs 6-MP and 6-TG.

It is an advantage of the present invention that the prodrugs of the invention exhibit less bone marrow and gastrointestinal toxicity than the thiopurine drugs.

### BRIEF DESCRIPTION OF THE SEVERAL VIEWS OF THE DRAWINGS

FIG. 1 depicts various thiopurine drugs, prodrugs and analogs.

FIG. 2 depicts urinary metabolites after separate administration of trans-AVTG, cis-AVTP and thiopurine 6-TG to mice.

### DETAILED DESCRIPTION OF THE INVENTION

The prodrugs trans-AVTG and cis-AVTP are more cytotoxic in vitro than the parent thiopurine drugs 6-TG and 6-MP, and more cytotoxic than AZA, a structural analog and prodrug of 6-MP that is currently used as an immunosuppressant. Without intending to be limited as to theory, it is believed that the increased cytotoxicity results from increased delivery by the prodrugs of the chemotherapeutic moiety into the cell as well as from perturbations in intracellular thiol homeostasis during prodrug metabolism. Also, the in vivo toxicity of the prodrugs is significantly less than that of equimolar doses of 6-TG.

Method for Synthesizing AVTP and AVTG

AVTG and AVTP were synthesized by dissolving 6-TG or 6-MP, respectively, in DMSO in a test tube at room temperature. Thereafter, approximately 3 equivalents of 3-butyn-2-one were added to the solution. The reaction was allowed to proceed for 10 min after which the reaction mixture was injected onto a Sephadex LH-20 column (Amersham Biosciences Inc., Piscataway, N.J.) for purification. The products were eluted with 10% acetonitrile in water adjusted to pH 2.5 with trifluoroacetic acid. Fractions containing cis- or trans-AVTG, or cis- or trans-AVTP were collected using a fraction collector, combined and lyophilized on a freeze dryer. The final products were greater than 96% pure as determined by HPLC with no trace of the parent thiopurine detected. While 96% or greater purity is sufficient for uses proposed in this application, further purification to, e.g., greater than 98%, and preferably greater than 99%, is feasible using conventional purification schemes. The identities of the prodrugs were confirmed by proton nuclear magnetic resonance and by electrospray ionization mass spectrometry. The prodrugs were stable as solids when refrigerated. Furthermore, the prodrugs are stable in solution for a few hours except when thiols are present, since the prodrugs react rapidly with sulfhydryl groups to form the parent thiopurine.

Under the reaction conditions used, the ratio of the trans to the cis isomers of AVTG formed is approximately 2:1, while the ratio of the cis to the trans isomers of AVTP formed is approximately 2:1. The isomer that was formed in greater quantity was used in our studies.

In Vitro Cytotoxicity of AVTP and AVTG

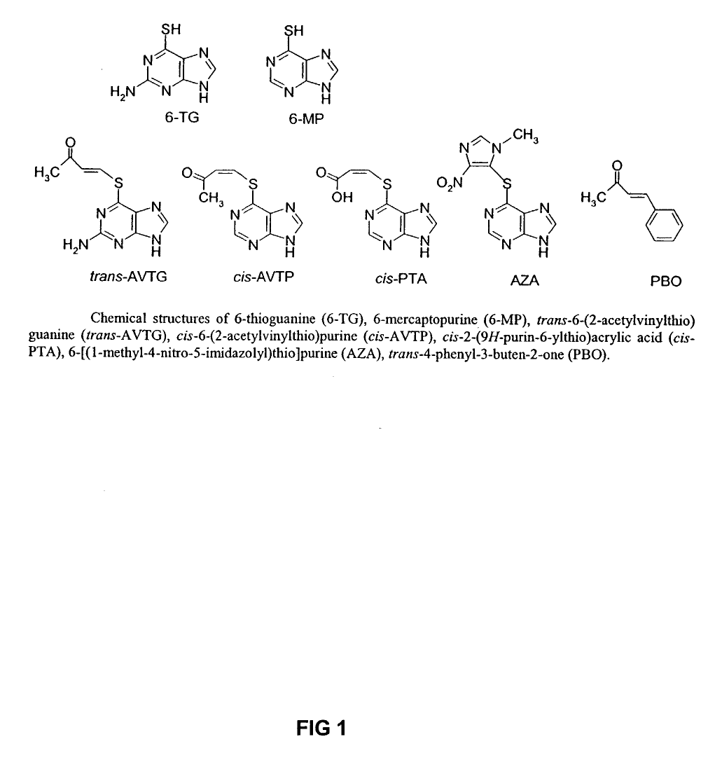

Preliminary studies examining the in vitro cytotoxicity of cis-AVTP and trans-AVTG as compared to the parent drugs, were carried out using the 3-[4,5-dimethylthiazol-2-yl]-2,5-diphenyl tetrazolium bromide (MTT) cytotoxicity assay. Table 1 shows the IC₅₀ values (the concentration where 50% reduction in color formation was observed for drug treated cells as compared with cells exposed to vehicle only) obtained after 72 h incubation of ACHN and A-498 cells with the prodrugs, the parent drugs, AZA and the structural analog phenylbutenone (PBO; FIG. 1) which reacts with GSH but does not release a thiopurine moiety.

TABLE 1

IC₅₀ (μM)

| Drug | ACHN | A-498 |
|---|---|---|
| 6-TG | 2.5 ± 0.8^{a} | 6.9 ± 4.1^{a} |
| 6-MP | 6.3 ± 1.9^{b} | 60.6 ± 25.5^{b} |
| trans-AVTG | 4.1 ± 0.4^{a,b} | 9.7 ± 2.0^{a} |
| cis-AVTP | 3.7 ± 0.7^{a} | 8.0 ± 1.2^{a} |
| AZA | 22.1 + 5.8^{c} | 50.2 ± 10.4^{b} |
| PBO | 62.3 ± 10.7^{d} | 252.5 ± 148.6^{c} |

No difference was observed between the IC₅₀ values of trans-AVTG and cis-AVTP in the cell lines tested. However, the IC₅₀ values for both prodrugs were approximately 2.5-fold higher for A-498 cells than for ACHN cells, which were more sensitive towards all the compounds tested. ACHN cells have a shorter doubling time than the A-498 cells (27.5 h vs 66.8 h, respectively) which may contribute in part to their higher sensitivity towards the drugs. It is worth pointing out that the IC₅₀ value for cis-AVTP was significantly lower than what was observed for the parent thiopurine 6-MP in the two cell lines tested. On the other hand, no difference in potency was observed between trans-AVTG and 6-TG in either cell line after 72 h drug incubation.

The finding that PBO was significantly less cytotoxic than trans-AVTG and cis-AVTP suggests that decreasing or depleting intracellular GSH is not the only determinant of cytotoxicity of the thiopurine prodrugs. Furthermore, the IC₅₀ value for AZA was 5- to 6-fold higher than the IC₅₀ values for trans-AVTG and cis-AVTP, while the propenoic acid analog PTA was not cytotoxic within the concentration range examined. It is of considerable interest to point out that the order of cytotoxicity of these thiopurine prodrugs is the same as their order of reactivity towards GSH.

Because cis-AVTP was significantly more cytotoxic than 6-MP after 72 h drug incubation, we examined the cytotoxicities of cis-AVTP and 6-MP at different time points. Lower IC₅₀ values were observed for cis-AVTP as compared with 6-MP in ACHN cells after 24, 48 and 72 h drug incubations while in the A-498 cell line, the IC₅₀ values obtained for cis-AVTP were significantly lower than those obtained for 6-MP at all the time points examined, as Table 2 demonstrates.

TABLE 2

| Cell line | h | 6-MP | cis-AVTP |
|---|---|---|---|
| ACHN | 24 | N.D. | 58.2 ± 1.7^{a} |
| | 48 | 44.8 ± 5.0^{a} | 8.7 ± 0.9^{b,♯} |
| | 72 | 6.3 ± 1.9^{b} | 3.7 ± 0.7^{c,♯} |
| | 96 | 0.9 ± 0.3^{c} | 3.5 ± 0.4^{c,♯} |
| A-498 | 24 | N.D. | 106.9 ± 14.7^{a} |
| | 48 | N.D. | 24.6 ± 4.0^{b} |
| | 72 | 60.6 ± 25.5^{a} | 8.0 ± 1.2^{c,♭} |
| | 96 | 14.9 ± 6.7^{b} | 5.5 ± 0.9^{d,♭} |

N.D. Value could not be determined due to limited cytotoxicity observed.
♯Significantly different from the IC₅₀ value of 6-MP in ACHN cells at the same time point (* p < 0.05).
♭Significantly different from the IC₅₀ value of 6-MP in A-498 cells at the same time point (* p < 0.05).

cis-AVTP and trans-AVTG were tested in National Cancer Institute's (NCI) 60 cell lines derived from leukemia, melanoma, lung, kidney cancers. Most chemotherapeutic agents currently nd 6-TG, have been tested in this screen and a database he National Institutes of Health. The NCI assigned s-AVTP, and number NSC-722289 to trans-AVTG. tocol used for the cytotoxicity assessment in the NCI e from the National Institutes of Health. Briefly, tumor ma, lung, colon, brain, ovary, breast, prostate, and um containing 5% fetal bovine serum and 2 mM L-glutamine re plated in a 100 μL volume into 96 well microtiter 37° C. in humidified atmosphere supplemented with 5% ach cell line were fixed with trichloroacetic acid for f drug addition (Tz). The drugs, as 400-fold concentrated uted to twice the final maximum test concentration with entamicin. Additional four 10-fold dilutions were made us control. Aliquots (100 μl) of the drug solutions were 100 μl of medium, resulting in the required final drug the plates were incubated at 37° C. in humidified for additional 48 h. The assay was terminated by the w/v) trichloroacetic acid for adherent and suspension minutes at 4° C. The supernatant was discarded, the and air dried. A 100 μl aliquot of 0.4% (w/v) sulforhodamine B (SRB) solution in 1% acetic acid was added to each well, and the plates incubated for 10 minutes at room temperature. After staining, unbound dye was removed by washing five times with 1% acetic acid and the plates air dried. Bound stain was solubilized with 10 mM trizma base, and the absorbance read on a plate reader at a wavelength of 515 nm.

By using time zero (Tz), control growth (C), and test growth in the presence of drug at the five concentration levels (Ti), the percentage growth was calculated at each of the drug concentration levels. Percentage growth inhibition was calculated as:

[(Ti−Tz)/(C−Tz)]×100 for concentrations for which Ti>/=Tz

[(Ti−Tz)/Tz]×100 for concentrations for which Ti<Tz.

Three dose response parameters were calculated for each experimental agent. The GI₅₀ (growth inhibition of 50%) was calculated from [(Ti−Tz)/(C−Tz)]×100=50, which is the drug concentration resulting in a 50% reduction in the net protein increase (as measured by SRB staining) in control cells during the drug incubation. The TGI, the drug concentration resulting in total growth inhibition was calculated from Ti=Tz. The LC₅₀ (the concentration of drug resulting in a 50% reduction in the measured protein at the end of the drug treatment as compared to that at the beginning) indicating a net loss of cells following treatment was calculated from [(Ti−Tz)/Tz]×100=−50. Values were calculated for each of these three parameters if the level of activity is reached; however, if the effect was not reached or was exceeded, the value for that parameter is expressed as greater or less than the maximum or minimum concentration tested. For assessment of cis-AVTP and trans-AVTG cytotoxicity, the lowest and highest drug dilution used were 0.01 μM and 100 μM, respectively, whereas the standard agent database indicated that the highest drug dilution used to assess the cytotoxicity of 6-MP and 6-TG was higher than 100 μM. However, in order to simplify the comparison between the prodrugs and the parent drugs, any GI₅₀, TGI, and LC₅₀ value obtained for 6-MP and 6-TG that was above 100 μM is listed as >100.

The in vitro cytotoxicity parameters GI₅₀, TGI, and LC₅₀ obtained for cis-AVTP and trans-AVTG in the NCI's anticancer screening program, are listed in Tables 3 and 4, respectively. Additionally, these same in vitro cytotoxicity parameters previously obtained for the parent thiopurines 6-MP and 6-TG are listed in Tables 5 and 6, respectively. Examination of the toxicity parameters revealed that cis-AVTP had GI₅₀, TGI, and LC₅₀ values lower than or equal to those of 6-MP in all the cell lines used to test the cytotoxicity of cis-AVTP.

TABLE 3
The parameters GI₅₀, TGI and LC₅₀ obtained for cis-AVTP in the NCI
anticancer screen. Parameters above or below the highest or lowest drug
concentration used are listed as >100 or <0.01, respectively.
All values are given in μM.

| Cell Line | Tissue type | GI₅₀ | TGI | LC₅₀ | Cell Line | Tissue type | GI₅₀ | TGI | LC₅₀ |
|---|---|---|---|---|---|---|---|---|---|
| NCI/ADR-RES | Breast | 0.51 | 2.79 | >100 | LOX IMVI | Melanoma | 0.03 | 2.23 | >100 |
| MDA-MB-231/ATCC | Breast | 1.27 | 4.56 | >100 | MALME-3M | Melanoma | N.D. | N.D. | N.D. |
| HS 578T | Breast | 0.46 | 8.79 | >100 | M14 | Melanoma | 0.07 | 1.89 | 7.75 |
| MDA-MB-435 | Breast | 0.56 | 2.75 | 9.80 | SK-MEL-2 | Melanoma | 0.48 | 8.22 | 40.7 |
| MDA-N | Breast | 0.55 | 4.48 | >100 | SK-MEL-28 | Melanoma | 1.84 | 4.19 | 9.58 |
| BT-549 | Breast | 1.24 | 3.48 | 9.78 | SK-MEL-5 | Melanoma | 1.30 | 3.24 | 8.07 |
| | | | | | UACC-257 | Melanoma | 2.40 | 27.4 | >100 |
| IGR-OV1 | Ovarian | 2.00 | 7.72 | 29.8 | UACC-62 | Melanoma | 0.23 | 1.10 | 5.88 |
| OVCAR-3 | Ovarian | 0.03 | 0.47 | 8.67 | | | | | |
| OVCAR-4 | Ovarian | 0.51 | 4.06 | 25.6 | A549/ATCC | Non-Small Cell Lung | 6.94 | 24.3 | 69.5 |
| OVCAR-5 | Ovarian | 1.31 | 2.94 | 6.57 | HOP-62 | Non-Small Cell Lung | 0.40 | 21.2 | 88.9 |
| OVCAR-8 | Ovarian | 1.53 | 5.93 | >100 | HOP-92 | Non-Small Cell Lung | 1.69 | 5.45 | 32.9 |
| SK-OV-3 | Ovarian | 0.11 | 5.04 | 33.3 | NCI-H23 | Non-Small Cell Lung | 1.48 | 4.58 | 27.8 |
| | | | | | NCI-H322M | Non-Small Cell Lung | 2.49 | 15.6 | 68.8 |
| PC-3 | Prostate | 2.05 | 10.2 | 85.1 | NCI-H460 | Non-Small Cell Lung | 0.68 | 8.71 | 38.6 |
| DU-145 | Prostate | 1.32 | 3.97 | 24.4 | NCI-H522 | Non-Small Cell Lung | N.D. | N.D. | N.D. |
| COLO 205 | Colon | 0.46 | 1.70 | 4.74 | SF-268 | CNS | <0.01 | 0.24 | 27.7 |
| HCT-116 | Colon | 0.51 | 1.89 | 4.73 | SF-295 | CNS | 0.56 | 11.2 | 57.8 |
| HCT-15 | Colon | 1.88 | 5.98 | 41.3 | SF-539 | CNS | 0.65 | 2.86 | 11.8 |
| HT29 | Colon | 0.73 | 2.85 | 9.23 | SNB-19 | CNS | 3.66 | 18.2 | 76.3 |
| KM12 | Colon | 3.35 | 27.2 | >100 | SNB-75 | CNS | 4.19 | 18.6 | 49.1 |
| SW-620 | Colon | 1.52 | >100 | >100 | U251 | CNS | 2.74 | 10.2 | 56.3 |
| 786-0 | Renal | 0.37 | 3.48 | 24.9 | CCRF-CEM | Leukemia | 0.20 | 2.17 | >100 |
| ACHN | Renal | 1.99 | 3.89 | 7.59 | HL-60(TB) | Leukemia | 0.49 | 4.95 | >100 |
| CAKI-1 | Renal | 0.11 | 0.55 | 2.85 | K-562 | Leukemia | 0.09 | >100 | >100 |
| SN12C | Renal | 2.03 | 5.75 | 28.1 | MOLT-4 | Leukemia | 0.36 | >100 | >100 |
| TK-10 | Renal | 0.03 | 0.64 | 13.9 | RPMI-8226 | Leukemia | 0.12 | 1.67 | >100 |
| UO-31 | Renal | 0.60 | 1.85 | 4.48 | SR | Leukemia | 0.17 | >100 | >100 |

N.D. cis-AVTP cytotoxicity was not determined in this cell line.

TABLE 4
The parameters GI₅₀, TGI and LC₅₀ obtained for trans-AVTG in the NCI
anticancer screen. Parameters above or below the highest or lowest drug
concentration used are listed as >100 or <0.01, respectively.
All values are given in μM.

| Cell Line | Tissue type | GI₅₀ | TGI | LC₅₀ | Cell Line | Tissue type | GI₅₀ | TGI | LC₅₀ |
|---|---|---|---|---|---|---|---|---|---|
| NCI/ADR-RES | Breast | 0.35 | 7.40 | >100 | LOX IMVI | Melanoma | 0.20 | 2.39 | 82.5 |
| MDA-MB-231/ATCC | Breast | 0.89 | 23.6 | >100 | MALME-3M | Melanoma | 0.37 | 3.07 | 29.3 |
| HS 578T | Breast | 1.78 | 26.9 | >100 | M14 | Melanoma | 0.45 | 12.0 | 53.5 |
| MDA-MB-435 | Breast | 1.04 | 12.9 | 51.5 | SK-MEL-2 | Melanoma | 1.02 | 15.5 | 98.7 |
| MDA-N | Breast | 1.28 | 10.3 | 76.2 | SK-MEL-28 | Melanoma | 11.6 | 24.8 | 52.7 |
| BT-549 | Breast | 0.39 | 5.75 | 43.1 | SK-MEL-5 | Melanoma | 1.68 | 8.10 | 41.7 |
| | | | | | UACC-257 | Melanoma | 11.6 | >100 | >100 |
| IGR-OV1 | Ovarian | 0.03 | 0.08 | 7.86 | UACC-62 | Melanoma | 0.28 | 0.93 | 24.9 |
| OVCAR-3 | Ovarian | 0.24 | 0.63 | 12.1 | | | | | |
| OVCAR-4 | Ovarian | 2.46 | 17.0 | 45.1 | A549/ATCC | Non-Small Cell Lung | 5.75 | 32.8 | >100 |
| OVCAR-5 | Ovarian | 0.54 | 6.04 | 57.7 | HOP-62 | Non-Small Cell Lung | 1.62 | >100 | >100 |
| OVCAR-8 | Ovarian | 0.55 | >100 | >100 | HOP-92 | Non-Small Cell Lung | 3.01 | 19.4 | 50.0 |
| SK-OV-3 | Ovarian | 0.24 | 0.78 | >100 | NCI-H23 | Non-Small Cell Lung | 3.05 | 16.3 | 61.5 |
| | | | | | NCI-H322M | Non-Small Cell Lung | 2.52 | 22.2 | >100 |
| PC-3 | Prostate | 3.96 | 19.7 | 62.5 | NCI-H460 | Non-Small Cell Lung | 0.59 | 3.57 | 49.4 |
| DU-145 | Prostate | N.D. | N.D. | N.D. | NCI-H522 | Non-Small Cell Lung | 0.02 | 0.05 | 8.28 |
| COLO 205 | Colon | 1.87 | 3.98 | 8.43 | SF-268 | CNS | <0.01 | 0.43 | 24.4 |
| HCT-116 | Colon | 0.67 | 3.01 | 14.7 | SF-295 | CNS | 0.43 | 0.92 | 82.9 |
| HCT-15 | Colon | 1.73 | 19.4 | >100 | SF-539 | CNS | 0.81 | 16.3 | >100 |
| HT29 | Colon | 1.06 | 12.5 | 60.4 | SNB-19 | CNS | 18.7 | 50.9 | >100 |
| KM12 | Colon | 2.71 | 13.0 | 64.8 | SNB-75 | CNS | 9.17 | >100 | >100 |
| SW-620 | Colon | 1.13 | 6.50 | >100 | U251 | CNS | 6.55 | 48.4 | >100 |
| 786-0 | Renal | 0.98 | 21.9 | >100 | CCRF-CEM | Leukemia | 0.14 | 1.83 | 54.8 |
| ACHN | Renal | 3.63 | 51.4 | >100 | HL-60(TB) | Leukemia | 3.77 | 16.6 | >100 |
| CAKI-1 | Renal | 0.16 | 0.51 | 13.7 | K-562 | Leukemia | 0.50 | >100 | >100 |
| SN12C | Renal | 2.84 | 14.3 | 52.8 | MOLT-4 | Leukemia | 0.70 | >100 | >100 |
| TK-10 | Renal | N.D. | N.D. | N.D. | RPMI-8226 | Leukemia | 0.24 | 3.19 | >100 |
| UO-31 | Renal | 0.62 | 11.9 | 39.1 | SR | Leukemia | 0.09 | 10.2 | >100 |

N.D. trans-AVTG cytotoxicity was not determined in this cell line.

TABLE 5
The parameters GI₅₀, TGI and LC₅₀ obtained for 6-MP in the NCI
anticancer screen. Parameters above or below the highest or lowest drug
concentration used are listed as >100 or <0.01, respectively.
All values are given in μM.

| Cell Line | Tissue type | GI₅₀ | TGI | LC₅₀ | Cell Line | Tissue type | GI₅₀ | TGI | LC₅₀ |
|---|---|---|---|---|---|---|---|---|---|
| NCI/ADR-RES | Breast | 3.18 | >100 | >100 | LOX IMVI | Melanoma | 0.35 | >100 | >100 |
| MDA-MB-231/ATCC | Breast | 16.4 | >100 | >100 | MALME-3M | Melanoma | 4.28 | >100 | >100 |
| HS 578T | Breast | 15.7 | >100 | >100 | M14 | Melanoma | 0.67 | >100 | >100 |
| MDA-MB-435 | Breast | 1.40 | >100 | >100 | SK-MEL-2 | Melanoma | 5.81 | >100 | >100 |
| MDA-N | Breast | 1.50 | >100 | >100 | SK-MEL-28 | Melanoma | >100 | >100 | >100 |
| BT-549 | Breast | >100 | >100 | >100 | SK-MEL-5 | Melanoma | 8.59 | >100 | >100 |
| | | | | | UACC-257 | Melanoma | 14.2 | >100 | >100 |
| IGR-OV1 | Ovarian | 8.59 | >100 | >100 | UACC-62 | Melanoma | 1.63 | >100 | >100 |
| OVCAR-3 | Ovarian | 0.71 | >100 | >100 | | | | | |
| OVCAR-4 | Ovarian | 7.11 | >100 | >100 | A549/ATCC | Non-Small Cell Lung | 27.7 | >100 | >100 |
| OVCAR-5 | Ovarian | 7.85 | >100 | >100 | HOP-62 | Non-Small Cell Lung | 1.69 | >100 | >100 |
| OVCAR-8 | Ovarian | 2.44 | >100 | >100 | HOP-92 | Non-Small Cell Lung | 2.60 | >100 | >100 |
| SK-OV-3 | Ovarian | 0.97 | >100 | >100 | NCI-H23 | Non-Small Cell Lung | 3.74 | >100 | >100 |
| | | | | | NCI-H322M | Non-Small Cell Lung | 7.69 | >100 | >100 |
| PC-3 | Prostate | 3.81 | >100 | >100 | NCI-H460 | Non-Small Cell Lung | 5.60 | >100 | >100 |
| DU-145 | Prostate | 1.73 | >100 | >100 | NCI-H522 | Non-Small Cell Lung | 1.67 | >100 | >100 |
| COLO 205 | Colon | 4.68 | >100 | >100 | SF-268 | CNS | 4.03 | >100 | >100 |
| HCT-116 | Colon | 2.33 | >100 | >100 | SF-295 | CNS | 5.36 | >100 | >100 |
| HCT-15 | Colon | 4.38 | >100 | >100 | SF-539 | CNS | 2.38 | >100 | >100 |
| HT29 | Colon | 3.97 | >100 | >100 | SNB-19 | CNS | >100 | >100 | >100 |
| KM12 | Colon | 7.31 | >100 | >100 | SNB-75 | CNS | 7.93 | >100 | >100 |
| SW-620 | Colon | 6.82 | >100 | >100 | U251 | CNS | 11.8 | >100 | >100 |
| 786-0 | Renal | 1.87 | >100 | >100 | CCRF-CEM | Leukemia | 1.22 | >100 | >100 |
| ACHN | Renal | 6.30 | >100 | >100 | HL-60(TB) | Leukemia | 2.30 | >100 | >100 |
| CAKI-1 | Renal | 2.36 | >100 | >100 | K-562 | Leukemia | 0.35 | >100 | >100 |
| SN12C | Renal | 22.6 | >100 | >100 | MOLT-4 | Leukemia | 1.51 | >100 | >100 |
| TK-10 | Renal | 1.83 | >100 | >100 | RPMI-8226 | Leukemia | 1.75 | >100 | >100 |
| UO-31 | Renal | 6.52 | >100 | >100 | SR | Leukemia | 1.27 | >100 | >100 |

TABLE 6
The parameters GI₅₀, TGI and LC₅₀ obtained for 6-TG in the NCI
anticancer screen. Parameters above or below the highest or lowest drug
concentration used are listed as >100 or <0.01, respectively.
All values are given in μM.

| Cell Line | Tissue type | GI₅₀ | TGI | LC₅₀ | Cell Line | Tissue type | GI₅₀ | TGI | LC₅₀ |
|---|---|---|---|---|---|---|---|---|---|
| NCI/ADR-RES | Breast | 0.59 | 35.6 | >100 | LOX IMVI | Melanoma | 0.21 | 8.99 | >100 |
| MDA-MB-231/ATCC | Breast | 1.94 | >100 | >100 | MALME-3M | Melanoma | 1.34 | 28.6 | >100 |
| HS 578T | Breast | 7.26 | >100 | >100 | M14 | Melanoma | 0.58 | 14.3 | >100 |
| MDA-MB-435 | Breast | 0.84 | 31.9 | >100 | SK-MEL-2 | Melanoma | 0.92 | 10.5 | >100 |
| MDA-N | Breast | 1.01 | 26.4 | >100 | SK-MEL-28 | Melanoma | 8.91 | >100 | >100 |
| BT-549 | Breast | 3.59 | >100 | >100 | SK-MEL-5 | Melanoma | 3.53 | 16.4 | 88.1 |
| | | | | | UACC-257 | Melanoma | 1.98 | 95.7 | >100 |
| IGR-OV1 | Ovarian | 4.79 | >100 | >100 | UACC-62 | Melanoma | 0.50 | 4.53 | 74.1 |
| OVCAR-3 | Ovarian | 0.71 | 6.68 | >100 | | | | | |
| OVCAR-4 | Ovarian | 1.49 | 99.5 | >100 | A549/ATCC | Non-Small Cell Lung | 3.48 | >100 | >100 |
| OVCAR-5 | Ovarian | 0.65 | 47.5 | >100 | HOP-62 | Non-Small Cell Lung | 0.73 | 28.4 | >100 |
| OVCAR-8 | Ovarian | 0.69 | 60.0 | >100 | HOP-92 | Non-Small Cell Lung | 0.73 | 24.0 | >100 |
| SK-OV-3 | Ovarian | 0.54 | 16.4 | >100 | NCI-H23 | Non-Small Cell Lung | 1.06 | 22.9 | >100 |
| | | | | | NCI-H322M | Non-Small Cell Lung | 7.33 | >100 | >100 |
| PC-3 | Prostate | 1.77 | >100 | >100 | NCI-H460 | Non-Small Cell Lung | 0.66 | 20.0 | >100 |
| DU-145 | Prostate | 0.78 | >100 | >100 | NCI-H522 | Non-Small Cell Lung | 0.91 | 6.28 | >100 |
| COLO 205 | Colon | 1.67 | 8.75 | 34.5 | SF-268 | CNS | 1.14 | >100 | >100 |
| HCT-116 | Colon | 0.53 | 16.2 | >100 | SF-295 | CNS | 1.02 | >100 | >100 |
| HCT-15 | Colon | 1.07 | 50.9 | >100 | SF-539 | CNS | 1.02 | 68.4 | >100 |
| HT29 | Colon | 1.13 | >100 | >100 | SNB-19 | CNS | 78.5 | >100 | >100 |
| KM12 | Colon | 1.72 | 31.5 | >100 | SNB-75 | CNS | 1.42 | 41.3 | >100 |
| SW-620 | Colon | 1.52 | >100 | >100 | U251 | CNS | 4.89 | >100 | >100 |
| 786-0 | Renal | 0.82 | >100 | >100 | CCRF-CEM | Leukemia | 0.15 | 25.4 | >100 |
| ACHN | Renal | 1.93 | >100 | >100 | HL-60(TB) | Leukemia | 1.07 | 17.8 | >100 |
| CAKI-1 | Renal | 0.53 | 15.6 | >100 | K-562 | Leukemia | 0.40 | 70.8 | >100 |
| SN12C | Renal | 1.25 | >100 | >100 | MOLT-4 | Leukemia | 0.27 | 15.4 | >100 |
| TK-10 | Renal | 1.04 | 78.3 | >100 | RPMI-8226 | Leukemia | 0.39 | 20.9 | >100 |
| UO-31 | Renal | 1.48 | 32.3 | >100 | SR | Leukemia | 0.19 | 17.9 | >100 |

Furthermore, cis-AVTP had lower GI₅₀, TGI, and LC₅₀ values than trans-AVTG in 35, 40, and 42 cell lines, respectively, of the 49 cell lines that were common to cis-AVTP and trans-AVTG cytotoxicity assessment. However, trans-AVTG had GI₅₀, TGI, and LC₅₀ values lower than or equal to those of 6-TG in 26, 44, and 51 cell lines, respectively, of the 51 cell lines used to assess trans-AVTG cytotoxicity. Cell lines that are especially sensitive toward cis-AVTP treatment are the breast cancer cell lines MDA-MB-435 and BT-549, the ovarian cancer cell lines OVCAR-3 and OVCAR-5, the colon cancer cell lines COLO 205, HCT-116, and HT29, the renal cancer cell lines ACHN, CAKI-1, TK-10, and UO-31, the melanoma cell lines M 14, SK-MEL-28, SK-MEL-5, and UACC-62, and the CNS cancer cell line SF-539. All of these 16 cell lines have GI₅₀, TGI, and LC₅₀ values below 2, 5, and 15 μM, respectively. Additionally, the leukemia cell lines CCRF-CEM, HL-60(TB), and RPMI-8226 have GI₅₀ and TGI values below 0.5 and 5 μM respectively. Cell lines that are especially sensitive toward trans-AVTG treatment are the ovarian cancer cell lines IGR-OV1 and OVCAR-3, the colon cancer cell lines COLO 205 and HCT-116, the renal cancer cell line CAKI-1, and the non-small cell lung cancer cell line NCI-H5222. These 6 cell lines also have GI₅₀, TGI, and LC₅₀ values below 2, 5, and 15 μM, respectively. Furthermore, the leukemia cell lines CCRF-CEM and RMPI-8226 show good response toward trans-AVTG treatment.

When the responses obtained from all the cell lines included in the screen are examined, as in Table 7, it is revealed that the median GI₅₀ value obtained for cis-AVTP is less than ⅕ of that obtained for 6-MP. Similarly, the median GI₅₀ value for cis-AVTP is only about 60% of that obtained for trans-AVTG. However, the median GI₅₀ value obtained for trans-AVTG is comparable to that obtained for the parent compound 6-TG. When the parameter TGI is examined, it is observed that the median TGI value for cis-AVTP is considerably lower than that obtained for 6-MP and less than half of that obtained for trans-AVTG. Unlike what was observed for the parameter GI₅₀, the median TGI value obtained for trans-AVTG is considerably less than that obtained for 6-TG. Similar results were obtained for the parameter LC₅₀ as were obtained for the parameter TGI.

TABLE 7
The median GI₅₀, TGI, and LC₅₀ values obtained for cis-AVTP,
trans-AVTG, 6-MP, and 6-TG in the NCI anticancer screen.
All values are in μM.

| Parameter | cis-AVTP | trans-AVTG | 6-MP | 6-TG |
|---|---|---|---|---|
| GI₅₀ | 0.60 | 0.98 | 3.97 | 1.04 |
| TGI | 4.56 | 12.9 | >100 | 41.3 |
| LC₅₀ | 38.6 | 76.2 | >100 | >100 |

To assess whether cells of different tissue types exhibit different sensitivity toward the prodrugs cis-AVTP and trans-AVTG, the cell lines included in NCI's screening panel were grouped together according to their tissue types and the median GI₅₀, TGI, and LC₅₀ values obtained for each tissue type were compared. The same was done for the parent thiopurines 6-MP and 6-TG. The median GI₅₀ value obtained for cis-AVTP for the various tissue types was in all cases lower than the median GI₅₀ values obtained for the parent thiopurine 6-MP (Tables 8A and 8B). Examination of the median GI₅₀ values obtained after cis-AVTP treatment revealed that leukemic cells were by far the most sensitive, whereas CNS- and prostate-derived cells were least sensitive toward cis-AVTP treatment (Tables 8A and 8B). Contrary to what was observed after cis-AVTP treatment, only 4 of the 9 tissue types examined had lower median GI₅₀ values after trans-AVTG treatment than 6-TG treatment. Leukemic cells as well as cells of ovarian origin were most sensitive toward trans-AVTG treatment whereas CNS- and prostate-derived cells were the least sensitive. Examination of the median GI₅₀ values obtained for 6-MP and 6-TG revealed that the thiopurines were also most effective against leukemic cells whereas breast cancer cells were the least sensitive (Tables 8A and 8B).

TABLE 8A
The median GI₅₀ values obtained within each tissue type
after cis-AVTP, trans-AVTG, 6-MP, or 6-TG treatment.
All values are in μM.

| Tissue type | cis-AVTP | trans-AVTG | 6-MP | 6-TG |
|---|---|---|---|---|
| Breast | 0.55 | 0.97 | 9.42 | 1.48 |
| Ovarian | 0.91 | 0.39 | 4.77 | 0.70 |
| Prostate | 1.69 | 3.96 | 2.77 | 1.27 |
| Colon | 1.13 | 1.43 | 4.53 | 1.32 |
| Renal | 0.49 | 0.98 | 4.33 | 1.14 |
| Melanoma | 0.48 | 0.73 | 5.04 | 1.13 |
| NSCL | 1.59 | 2.52 | 3.74 | 0.91 |
| CNS | 1.69 | 3.68 | 6.64 | 1.28 |
| Leukemia | 0.19 | 0.37 | 1.39 | 0.33 |

TABLE 8B
The median GI₅₀ values obtained within each tissue
type after cis-AVTP, trans-AVTG, 6-MP, or 6-TG treatment.
For each compound, the tissues are ranked from the most
sensitive to the least sensitive. All values are in μM.

| cis-AVTP | | trans-AVTG | | 6-MP | | 6-TG | |
|---|---|---|---|---|---|---|---|
| Leukemia | 0.19 | Leukemia | 0.37 | Leukemia | 1.39 | Leu- | 0.33 |
| | | | | | | kemia | |
| Melanoma | 0.48 | Ovarian | 0.39 | Prostate | 2.77 | Ovarian | 0.70 |
| Renal | 0.49 | Melanoma | 0.73 | NSCL | 3.74 | NSCL | 0.91 |
| Breast | 0.55 | Breast | 0.97 | Renal | 4.33 | Mel- | 1.13 |
| | | | | | | anoma | |
| Ovarian | 0.91 | Renal | 0.98 | Colon | 4.53 | Renal | 1.14 |
| Colon | 1.13 | Colon | 1.43 | Ovarian | 4.77 | Prostate | 1.27 |
| NSCL | 1.59 | NSCL | 2.52 | Melanoma | 5.04 | CNS | 1.28 |
| Prostate | 1.69 | CNS | 3.68 | CNS | 6.64 | Colon | 1.32 |
| CNS | 1.69 | Prostate | 3.96 | Breast | 9.42 | Breast | 1.48 |

When similar examinations are carried out for the endpoint TGI, it is observed that the median TGI values obtained after cis-AVTP treatment are in all cases lower than those obtained after 6-MP treatment. The most and least sensitive tissue types toward cis-AVTP treatment are renal- and leukemia-derived cells, respectively. The median TGI values obtained after trans-AVTG treatment are similarly always lower than those obtained after 6-TG treatment (Tables 9A and 9B). In general, the most sensitive cell lines are of ovarian origin, whereas CNS-derived cells are the least sensitive cell lines toward trans-AVTG treatment (Tables 9A and 9B). It is of interest to note that the median TGI values obtained after 6-MP treatment for all tissue types are above 100 μM whereas the only median TGI values above 100 μM after 6-TG treatment are observed in CNS- and prostate derived cells.

TABLE 9A
The median TGI values obtained within each tissue type
after cis-AVTP, trans-AVTG, 6-MP, or 6-TG treatment.
All values are in μM.

| Tissue type | cis-AVTP | trans-AVTG | 6-MP | 6-TG |
|---|---|---|---|---|
| Breast | 3.98 | 11.6 | >100 | 67.8^{#} |
| Ovarian | 4.55 | 3.41 | >100 | 53.8 |
| Prostate | 7.09 | 19.7 | >100 | >100 |
| Colon | 4.42 | 9.50 | >100 | 41.2 |
| Renal | 2.67 | 14.3 | >100 | 89.2^{#} |
| Melanoma | 3.24 | 10.1 | >100 | 15.3 |
| NSCL | 12.2 | 19.4 | >100 | 24.0 |
| CNS | 10.7 | 32.4 | >100 | >100 |
| Leukemia | 52.5^{#} | 13.4 | >100 | 19.4 |

^{#} This median value is derived from three exact values below 100 μM and three values anywhere above 100 μM; this value may therefore be an underestimation.

TABLE 9B
The median TGI values obtained within each tissue
type after cis-AVTP, trans-AVTG, 6-MP, or 6-TG treatment.
For each compound, the tissues are ranked from the most
sensitive to the least sensitive. All values are in μM.

| cis-AVTP | | trans-AVTG | | 6-MP | | 6-TG | |
|---|---|---|---|---|---|---|---|
| Renal | 2.67 | Ovarian | 3.41 | Breast | >100 | Mel- | 15.3 |
| | | | | | | anoma | |
| Melanoma | 3.24 | Colon | 9.50 | CNS | >100 | Leu- | 19.4 |
| | | | | | | kemia | |
| Breast | 3.98 | Melanoma | 10.1 | Colon | >100 | NSCL | 24.0 |
| Colon | 4.42 | Breast | 11.6 | Leukemia | >100 | Colon | 41.2 |
| Ovarian | 4.55 | Leukemia | 13.4 | Melanoma | >100 | Ovarian | 53.8 |
| Prostate | 7.09 | Renal | 14.3 | NSCL | >100 | Breast | 67.8^{#} |
| CNS | 10.7 | NSCL | 19.4 | Ovarian | >100 | Renal | 89.2^{#} |
| NSCL | 12.2 | Prostate | 19.7 | Prostate | >100 | CNS | >100 |
| Leukemia | 52.5^{#} | CNS | 32.4 | Renal | >100 | Prostate | >100 |

^{#}This median value is derived from three exact values below 100 μM and three values anywhere above 100 μM; this value may therefore be an underestimation.

When the median LC₅₀ values obtained for each tissue are examined, it is observed that for all the tissue types, the median LC₅₀ value obtained after cis-AVTP or trans-AVTG treatment are equal to or lower than those obtained after treatment with the corresponding parent thiopurine (Tables 10A and 10B). It is of interest to note that the cellular response toward cis-AVTP is stepwise and can be grouped into four groups; in the first group are melanoma and renal cancer cells that are by far the most sensitive toward cis-AVTP treatment; in the second group are colon and ovarian cancer cells that are somewhat less sensitive; in the third group are cells of CNS, lung, and prostate origin that are not very sensitive toward cis-AVTP treatment whereas the cell lines in the fourth group, leukemic and breast cancer cells, are not responsive toward cis-AVTP (Tables 10A and 10B). This type of response is not observed after treatment with trans-AVTG where all the cell lines have LC₅₀ values above 50 μM. It is also of interest to note that after treatment with 6-MP and 6-TG, none of the tissue types have median LC₅₀ values under 100 μM.

TABLE 10A
The median LC₅₀ values obtained within each tissue type
after cis-AVTP, trans-AVTG, 6-MP, or 6-TG treatment.
All values are in μM.

| Tissue type | cis-AVTP | trans-AVTG | 6-MP | 6-TG |
|---|---|---|---|---|
| Breast | >100 | 88.1^{#} | >100 | >100 |
| Ovarian | 27.7 | 51.4 | >100 | >100 |
| Prostate | 54.8 | 62.5 | >100 | >100 |
| Colon | 25.3 | 62.6 | >100 | >100 |
| Renal | 10.7 | 52.8 | >100 | >100 |
| Melanoma | 9.58 | 53.1 | >100 | >100 |
| NSCL | 53.7 | 61.5 | >100 | >100 |
| CNS | 52.7 | >100 | >100 | >100 |
| Leukemia | >100 | >100 | >100 | >100 |

^{#}This median value is derived from three exact values below 100 μM and three values anywhere above 100 μM; this value may therefore be an underestimation.

TABLE 10B
The median LC₅₀ values obtained within each tissue
type after cis-AVTP, trans-AVTG, 6-MP, or 6-TG treatment.
For each compound, the tissues are ranked from the most
sensitive to the least sensitive. All values are in μM.

| cis-AVTP | | trans-AVTG | | 6-MP | | 6-TG | |
|---|---|---|---|---|---|---|---|
| Melanoma | 9.58 | Ovarian | 51.4 | Breast | >100 | Breast | >100 |
| Renal | 10.7 | Renal | 52.8 | CNS | >100 | CNS | >100 |
| Colon | 25.3 | Melanoma | 53.1 | Colon | >100 | Colon | >100 |
| Ovarian | 27.7 | NSCL | 61.5 | Leukemia | >100 | Leu- | >100 |
| | | | | | | kemia | |
| CNS | 52.7 | Prostate | 62.5 | Melanoma | >100 | Mel- | >100 |
| | | | | | | anoma | |
| NSCL | 53.7 | Colon | 62.6 | NSCL | >100 | NSCL | >100 |
| Prostate | 54.8 | Breast | 88.1^{#} | Ovarian | >100 | Ovarian | >100 |
| Breast | >100 | CNS | >100 | Prostate | >100 | Prostate | >100 |
| Leukemia | >100 | Leukemia | >100 | Renal | >100 | Renal | >100 |

^{#}This median value is derived from three exact values below 100 μM and three values anywhere above 100 μM; this value may therefore be an underestimation.

The cis-AVTP and trans-AVTG prodrugs are more cytotoxic in vitro than their parent thiopurines. cis-AVTP is equally or more toxic than its parent thiopurine in all the cell lines included in the screen for all the endpoints GI₅₀, TGI, and LC₅₀. Furthermore, the median GI₅₀, TGI, and LC₅₀ values for cis-AVTP were lower than those obtained for trans-AVTG and 6-TG. In light of the fact that cis-AVTP is a prodrug of the less toxic thiopurine, these findings are noteworthy. Similarly, trans-AVTG was in general more cytotoxic than its parent thiopurine 6-TG. It is possible that the higher intracellular thiopurine concentrations after prodrug treatment may play a role in their increased cytotoxicity. Additionally, intracellular metabolism of trans-AVTG to 6-TG leads to disruption of the cellular GSH homeostasis. It has been suggested that disruption of intracellular GSH status can affect the intracellular redox state and initiate apoptosis. Therefore, perturbation of intracellular GSH homeostasis may enhance the cytotoxic effects of the thiopurines.

It is of interest to point out that after treatment with cis-AVTP, leukemic cells had the lowest median GI₅₀ value but the highest the median TGI and LC₅₀ of all the tissue types included in the screen. Similar response was obtained after treatment with the parent thiopurine 6-MP; leukemic cells had the lowest median GI₅₀ value of the tissue types whereas the TGI and LC₅₀ values were above 100 μM. In light of the fact that 6-MP is successfully used as an antileukemic agent, these low GI₅₀ but high TGI and LC₅₀ values obtained for leukemic cells show that, at least in some cases, a clinically useful drug can have high TGI and LC₅₀ values.

After treatment with cis-AVTP, breast cancer cells exhibited very low median GI₅₀ and TGI values whereas their median LC₅₀ value was above 100 μM. Interestingly, even though the median LC₅₀ values were high, four of the cell lines had LC₅₀ values over 100 μM whereas two cell lines had LC₅₀ values below 10 μM. Thus, there appears to be a bimodal response toward cis-AVTP in the treatment of breast cancer cells when LC₅₀ is used as the endpoint, most likely because of as of yet unknown inherent differences among the breast cancer cell lines used in the screen.

The response of melanoma and renal cancer cell lines toward treatment with cis-AVTP is also of considerable significance. These two tissue types had the second and third lowest GI₅₀ values, and the lowest and second lowest TGI and LC₅₀ values, suggesting that they are very sensitive toward cis-AVTP treatment. The sensitivity of renal cancer cells is of importance because no chemotherapeutic agent is currently effective against renal cell carcinoma. At this point, the cause for the sensitivity toward cis-AVTP is unclear. Because cis-AVTP is a prodrug of 6-MP and because the cytotoxicity of 6-MP is believed to be, at least in part, due to the incorporation of faulty nucleotides into DNA, the cellular doubling time may play a role in the sensitivity of these tissue types toward cis-AVTP. However, the median doubling times of melanoma and renal cancer cell lines, 33.2 h and 34.3 h, respectively, are longer than the median doubling time (31.3 h) observed for all the cell lines included in the screen. Thus, it appears unlikely that doubling time is the major factor determining the sensitivity of melanoma and renal cancer cell lines toward cis-AVTP. Alternatively, these tissue types exhibit high intracellular GSH levels and may be capable of bioactivating cis-AVTP to a larger extent than other tissue types. However, the median GSH levels of melanoma and renal cancer cells, 14.9 and 2.8 nmol/mg protein, respectively, are above and below the median GSH value (13.0 nmol/mg protein) obtained for all the cell lines included in the screen. Thus, cellular GSH status alone cannot explain the sensitivity of these tissue types toward cis-AVTP. Therefore, it is most likely that an unknown cellular factor or factors, or a combination of multiple factors, contribute to the sensitivity of melanoma and renal cancer cell lines toward cis-AVTP.

The finding that the GI₅₀ and TGI values of trans-AVTG are comparable to or better than those of 6-TG, suggest that trans-AVTG may possess similar or better antileukemic activity than 6-TG. When the GI₅₀ values obtained after trans-AVTG and 6-TG treatment are examined, it is of interest to note that 5 of 6 ovarian cancer cell lines are more sensitive toward trans-AVTG than 6-TG whereas the opposite is true for colon cancer cells. These observations suggest that the mechanism of action of trans-AVTG and 6-TG is different.

In general, melanoma and ovarian cancer cells appear to be the most sensitive cell lines toward trans-AVTG treatment. The sensitivity of ovarian cancer cells toward trans-AVTG treatment is of importance because currently, there is a need for new chemotherapeutic drugs effective against ovarian cancers. Both the melanoma and ovarian panels have low GI₅₀ and TGI values whereas their LC₅₀ values are above 50 μM, possibly suggesting that trans-AVTG may act as a cytostatic compound rather than a cytotoxic compound in these cells. However, a trimodal response is seen when the LC₅₀ values for individual cell lines making up the panel of ovarian cells, is examined. Two cell lines have low LC₅₀ values (7.86 and 12.1 μM), two cell lines have LC50 values around 50 μM whereas two cell lines have LC₅₀ values above 100 μM. Thus, trans-AVTG may be useful against a subpopulation of ovarian cancers. The sensitivity of ovarian cancer cells toward trans-AVTG treatment is unlikely to be due to either short doubling time or high GSH levels; the panel of ovarian cell has a median doubling time of 38.1 h and median GSH content of 11.5 nmol/mg protein that are higher and lower, respectively, than the median doubling time and median GSH levels for all cell lines included in the screen, 31.3 h and 13.0 nmol/mg protein, respectively. Even the two ovarian cell lines that exhibit the lowest LC₅₀ values have doubling times that are similar to or longer than (31 h and 34.7 h) the median doubling time and GSH levels (9.3 and 9.8 nmol/mg protein) that are lower than the median GSH levels.

Taken together, these analyses suggest that cis-AVTP and trans-AVTG show both similar and distinct activities against the cell lines included in the NCI anticancer screen. Both compounds appear active against leukemias; this finding is consistent with the use of the parent thiopurines as antileukemic agents. Furthermore, melanoma cells appear relatively sensitive toward treatment with both compounds. However, cis-AVTP appears much more effective against renal cell lines whereas trans-AVTG is more effective against ovarian cancer cell lines. Examination of the sensitivity of individual cell lines revealed that the breast cancer lines MDA-MB-435 and BT-549, the ovarian cancer cell lines OVCAR-3 and OVCAR-5, the colon cancer cell lines COLO 205, HCT-116, and HT29, the renal cancer cell lines ACHN, CAKI-1, TK-10, and UO-31, the melanoma lines M14, SK-MEL-28, SK-MEL-5, and UACC-62, the CNS cancer cell line SF-539, and the leukemia cell lines CCRF-CEM, HL-60(TB), and RPMI-8226 all show good sensitivity toward cis-AVTP and might thus be interesting models in which to test the in vivo efficacy of cis-AVTP. Similarly, the ovarian cancer cell lines IGR-OV1 and OVCAR-3, the colon cancer cell lines COLO 205 and HCT-116, the renal cancer cell line CAKI-1, the non-small cell lung cancer cell line NCI-H5222, and the leukemia cell lines CCRF-CEM and RMPI-8226 show good sensitivity toward trans-AVTG treatment and might be interesting models for the in vivo efficacy testing of trans-AVTG.

In Vivo Metabolism and Toxicity

Thiopurine drugs have antitumor effect in human and non-human animals when administered at dosage levels and regimens which are known to be effective, but which are also known to cause toxicity to bone marrow in vivo. Comparable amounts of thiopurine prodrugs of the invention were administered to non-human animals in the following trials.

Heterozygous nude CD-1 male mice were treated (i.p.) with multiple doses consisting of either one dose daily for three consecutive days (cis-AVTP or trans-AVTG, 21.25 μmol/kg, 6-TG, 8.5 or 21.25 μmol/kg, or vehicle; one cycle), or one dose daily for three consecutive days, repeated again five days later (cis-AVTP, trans-AVTG or 6-TG, 8.5 or 21.25 μmol/kg, or vehicle; two cycles). These doses are comparable to doses of 6-TG that had anti-tumor activity against sarcoma 180 in mice.

The mice were sacrificed 24 h after the last treatment. Urine was collected for 24 h before initiation of treatment and every 24 h thereafter until the mice were sacrificed. Blood was collected by cardiac puncture and blood cells counted. Sections of liver, kidney, intestines, stomach and sternum were stained with hematoxylin and eosin and subjected to pathological analysis. Bone marrow smears were prepared for assessment of bone marrow cellularity. Liver function was evaluated by measuring alanine aminotransferase-(ALT) and aspartate aminotransferase (AST) activities in plasma. Kidney function was evaluated by measuring blood urea nitrogen in plasma and γ-glutamyltransferase activity and glucose levels in urine.

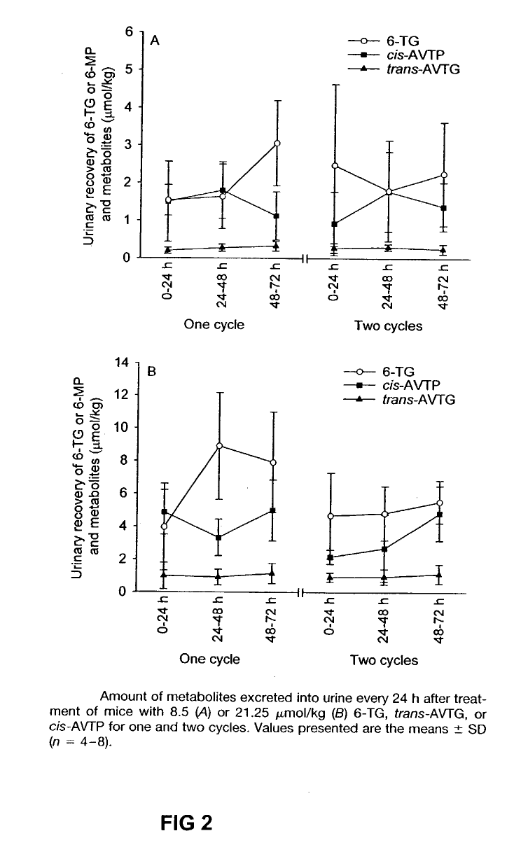

No change in the recovery of urinary metabolites was observed with increased dose when animals were treated with multiple doses of trans-AVTG, cis-AVTP and 6-TG. Approximately 17, 4 and 26% of the administered dose was recovered as thiopurines and further urinary metabolites after treatment with cis-AVTP, trans-AVTG and 6-TG, respectively (FIG. 2).

No change in peripheral white blood cell counts was seen after treatment with cis-AVTP, and only the 21.25 μmol/kg dose of trans-AVTG for two cycles decreased white cell counts. In contrast, mice treated with 8.5 or 21.25 μmol/kg 6-TG for one cycle or 21.25 μmol/kg for two cycles had reduced white blood cell counts (Table 11, Table 12, Table 13). However, the bone marrow myeloid to erythroid (“M:E”) ratios were 1.6:1, 1.8:1, 5.7:1 and 87.0:1, in mice receiving vehicle, 21.25 μmol/kg of cis-AVTP, trans-AVTG or 6-TG, respectively, for two cycles (Table 11).

TABLE 11
Hematology analyses after treatment of mice with
cis-AVTP, trans-AVTG or 6-TG for one
cycle. Superscript letters are comparisons between
cis-AVTP, trans-AVTG and 6-TG at the same
dose; values with different superscripts are significantly
different (p < 0.05). Values presented are
the mean ± S.D. (n = 3-4). WBC, white blood cells;
RBC, red blood cells; PLT, platelets.

| | Dose | | | |
|---|---|---|---|---|
| | (μmol/ | WBC | RBC | PLT |
| Drug | kg) | (× 10³ cells/μl) | (× 10⁶ cells/μl) | (× 10³ cells/μl) |
| Control | | 9.6 ± 2.3 | 9.5 ± 0.4 | 1134 ± 111 |
| cis-AVTP | 21.25 | 8.0 ± 1.2^{a} | 8.9 ± 0.6^{a} | 1644 ± 253^{a} |
| trans-AVTG | 21.25 | 7.4 ± 0.9^{a} | 9.1 ± 0.7^{a} | 1101 ± 383^{a,b} |
| 6-TG | 8.5 | 4.8 ± 1.2* | 8.3 ± 0.4* | 1172 ± 161 |
| | 21.25 | 3.8 ± 1.1^{b,}* | 8.4 ± 0.6^{a} | 752 ± 416^{b} |

*Significantly different from corresponding control (p < 0.05).

TABLE 12
Hematology analysis for mice treated with
vehicle alone (control), 6-TG, trans-AVTG, or cis-
AVTP for two cycles as described in Materials and Methods.
Values presented are the means ± S.D. (n = 3-4). WBC,
white blood cells; RBC, red blood cells; PLT, platelets.

| | Dose | | | |
|---|---|---|---|---|
| | (μmol/ | WBC | RBC | PLT |
| Drug | kg) | (× 10³ cells/μl) | (× 10⁶ cells/μl) | (× 10³ cells/μl) |
| Control | | 7.8 ± 1.1 | 9.3 ± 0.4 | 1167 ± 204 |
| 6-TG | 8.5 | 5.3 ± 1.6 | 8.3 ± 0.2 | 906 ± 140 |
| | 21.25 | 3.6 ± 0.1* | 8.0 ± 0.3* | 1351 ± 126 |
| trans-AVTG | 8.5 | 5.4 ± 1.6 | 8.9 ± 0.5 | 1054 ± 61 |
| | 21.25 | 2.3 ± 1.2* | 7.6 ± 1.0* | 828 ± 144 |
| cis-AVTP | 8.5 | 5.9 ± 2.3 | 9.5 ± 0.3 | 1160 ± 349 |
| | 21.25 | 8.4 ± 1.9 | 9.0 ± 0.5 | 1257 ± 335 |

*Significantly different from the corresponding control value (p < 0.05).

TABLE 13
Ratio of myeloid to erythroid cells (M:E ratio) in
bone marrow of mice treated with vehicle alone
(control), 6-TG, trans-AVTG, or cis-AVTP as
described in Materials and Methods. Values
presented are the means and range (n = 4).

| | |
|---|---|
| Dose | M:E ratio (mean and range) |

| | | |
|---|---|---|
| (μmol/kg) | One cycle | Two cycles |

| | | | |
|---|---|---|---|
| Control | | 1.7:1 (1.5:1-2.3:1) | 1.6:1 (1.2:1-1.8:1) |
| 6-TG | 8.5 | N.D. | 11.8:1 (8.1:1-15.7:1) |
| | 21.25 | 44.8:1 (32.3:1-49:1) | 86.5:1 (49:1-99:1) |
| trans-AVTG | 8.5 | N.D. | 3.2:1 (2.0:1-5.3:1) |
| | 21.25 | 6.6:1 (3.0:1-10.1:1) | 5.7:1 (4.6:1-7.3:1) |
| cis-AVTP | 8.5 | N.D. | 1.4:1 (1.2:1-1.6:1) |
| | 21.25 | 1.8:1 (1.5:1-2.3:1) | 1.8:1 (1.3:1-2.2:1) |

N.D. Not done

TABLE 14
Clinical parameters measuring liver function. Superscript letters
are comparisons between cis-AVTP, trans-AVTG and 6-TG
at the same dose and treatment length; values with different
superscripts are significantly different (p < 0.05).
Values presented are the means ± S.D. (n = 3-4).

| | Dose | AST (SF U/ml) | | ALT (SF U/ml) | |
|---|---|---|---|---|---|
| Drug | (μmol/kg) | One cycle | Two cycles | One cycle | Two cycles |
| Control | | 71.4 ± 44.9 | 40.2 ± 26.9 | 6.1 ± 2.5 | 8.1 ± 4.4 |
| Cis-AVTP | 8.5 | | 55.1 ± 37.5^{a} | | 7.8 ± 4.0^{a} |
| | 21.25 | 25.5 ± 4.3^{a} | 60.2 ± 30.8^{a} | 2.0 ± 2.0^{a,}* | 16.0 ± 4.3^{a,#} |
| trans-AVTG | 8.5 | | 43.6 ± 26.8^{a} | | 14.5 ± 5.0^{a} |
| | 21.25 | 132.5 ± 40.7^{b} | 236.9 ± 40.4^{b,#,}* | 46.3 ± 41.6^{b,}* | 20.7 ± 1.3^{a,}* |
| 6-TG | 8.5 | 43.5 ± 24.8 | 37.9 ± 12.2^{a} | 9.2 ± 3.2 | 10.5 ± 6.6^{a} |
| | 21.25 | 44.4 ± 5.4^{c} | 68.2 ± 54.5^{a} | 15.0 ± 2.5^{b} | 12.6 ± 4.8^{a} |

*Significantly different from the corresponding control value.
#Significantly different from the corresponding one cycle value.

These data suggest that 6-TG in particular, and trans-AVTG to a much lesser extent, have a more severe effect on erythroid and/or erythroid precursor cells than they do on white blood cells and their precursors. The change in peripheral white blood cell counts is more pronounced than the change in peripheral red blood cells because white blood cells have a much shorter lifespan than red blood cells. Interestingly, cis-AVTP does not affect the M:E ratio or peripheral blood cell counts.

In the small intestines, crypt epithelial cell apoptosis increased slightly in mice receiving multiple doses of cis-AVTP and extensively in mice treated with multiple doses of trans-AVTG or 6-TG. Similarly, in the large intestines, no increase or slight increase in crypt epithelial apoptosis was seen in mice treated with multiple doses of cis-AVTP and trans-AVTG, respectively, although considerable crypt cell apoptosis was detected in 6-TG treated mice.

No treatment group exhibited kidney toxicity as assessed by kidney function tests while liver function tests suggested a slight impairment of liver function in mice treated with trans-AVTG (Table 14). However, histopathological examination detected no liver lesions in this group, and no stomach or kidney lesions were detected in any treatment group.

In Vivo Tissue Distribution

Mice were injected (i.p.) with 42.5 μmol/kg cis-AVTP or trans-AVTG and were sacrificed after 15, 30 and 45 min. Blood was collected by cardiac puncture and metabolites in plasma and red blood cell fractions were quantified. Intestines and livers of treated mice were also harvested and analyzed for metabolites. All samples were analyzed before and after boing for 45 min to hydrolyze any nucleotides, nucleosides and conjugates that might have formed in vivo.

Analysis of tissue distributions of thiopurine, thiopurine riboside, thiopurine nucleotide, thioxanthine and thiouric acid metabolites revealed that the highest metabolite concentration in tissues was obtained at 15 min with lower concentrations detected at 30 and 45 min, except in the intestines where the concentration did not change much during the trial. The metabolite tissue concentrations at 15 min reached biologically relevant concentrations, even when the inactive metabolites thioxanthine and thiouric acid were not included in the analysis.

The data suggest that the metabolism of cis-AVTP and trans-AVTG is not saturated at the doses used. The significantly reduced metabolite excretion of trans-AVTG as compared with 6-TG may suggest a difference in cellular uptake of the two drugs. Furthermore, mice receiving multiple treatments with cis-AVTP or trans-AVTG at doses similar to those of 6-TG that were previously shown to have antitumor activity in mice, have significantly reduced intestinal and bone marrow toxicity as compared with mice treated with multiple doses of 6-TG. This is notable because bone marrow toxicity is the most common and limiting side effect associated with thiopurine use.

### PROPHETIC EXAMPLE

At least one prodrug of the invention is administered in a pharmaceutically acceptable carrier to a human or non-human animal having a solid or blood borne tumor, in a delivery regimen sufficient to reduce or prevent growth of the tumor. A suitable delivery regimen can include delivery of between 1 and 100 μmol of at least one of the prodrugs of the invention, or combination thereof per kilogram of the subject being treated. The prodrug(s) can be provided in a pharmaceutically acceptable carrier such as a buffered aqueous salt solution, e.g., phosphate buffered saline at physiological pH, or in a tablet comprising conventional binders and drug release agents as are known to the skilled artisan. The carrier should be free of sulfhydryl groups. The value of providing these compounds in combination with a suitable carrier has not previously been appreciated in the art, particularly when considering the unexpected reduction in bone marrow and intestinal toxicity in combination with enhanced tumor cell cytotoxicity. In fact, the prior art suggests that many compounds shown to be cytotoxic in vitro are ineffective in vivo because of their systemic toxicity or because of inadequate delivery of the active drug to tumor cells, thereby precluding the skilled artisan from predicting the in vivo performance of any given compound. The observations reported herein suggest a reasonable likelihood of efficacy in vivo without the detrimental adverse systemic effects.

The prodrugs should be stored as a solid and if administered in liquid form, should be solubilized shortly before administration (for example, within 2 hours, although longer times may be adequate). The prodrug(s) can be delivered by any pharmacologically acceptable route including but not limited to intravenous, intraperitoneal, or intramuscular injection, as well as oral or transdermal administration. The prodrug(s) can be delivered in a manner consistent with good medical practice, including but not limited to a plurality of spaced apart doses over the course of several days, for example in the manner described above in connection with in vivo toxicity and metabolism studies in an animal model. It is specifically contemplated that the skilled artisan can determine a suitable regimen using conventional methods for determining therapeutic dosages.

Upon examination after treatment according to the method, growth of the tumor is reduced or prevented. A solid tumor is preferably reduced in diameter by at least 10 percent. If not present on the skin of the patient, the tumor can be examined for change in diameter by surgical examination, or by any otherwise conventional method used to visualize internal solid tumors, including but not limited to radiologic, ultrasonic and magnetic resonance imaging methods, as appropriate. If blood borne, reduction in tumor growth can be observed using conventional methods. It is specifically contemplated that solid tumors comprising neoplastic cells from at least one of kidney, colon, ovary and skin respond to treatment according to the method of the invention, in view of the demonstrated ability of the prodrugs to effectively kill cell lines of these lineages in vitro.

We claim:

1. A prodrug comprising:
a pharmaceutically acceptable carrier; and
a thiopurine having a sulfur heteroatom conjugated to an alpha-, beta-unsaturated carbonyl moiety, the moiety comprising a double bond having an alpha end and a beta end, the beta end being accessible to glutathione in an addition-elimination reaction, the prodrug lacking an ionizable carboxylic acid group.

2. A prodrug as claimed in claim 1 wherein the moiety comprises at least three carbons.

3. A prodrug as claimed in claim 2 wherein the moiety comprises 4 carbons.

4. A prodrug as claimed in claim 1 wherein the moiety comprises a terminal carbon having an amine group attached thereto.

5. A prodrug as claimed in claim 1 wherein the thiopurine is selected from the group consisting of 6-thioguanine, 6-mercaptopurine, and a derivative of either of the foregoing.

6. A prodrug as claimed in claim 1 wherein the thiopurine is selected from the group consisting of 6-(2-acetylvinylthio)guanine (AVTG) and 6-(2-acetylvinylthio)purine (AVTP).

7. A method for treating a tumor in a tissue of a human or non-human animal, the tumor having an elevated level of glutathione relative to the tissue, the method comprising the steps of:
administering to the animal a prodrug comprising a thiopurine having a sulfur heteroatom conjugated to an alpha-, beta-unsaturated carbonyl moiety, in combination with a pharmaceutically acceptable carrier, the moiety comprising a double bond having an alpha end and a beta end, the beta end being accessible to glutathione in an addition-elimination reaction, the prodrug lacking an ionizable carboxylic acid group; and
observing a reduction in growth of the tumor.

8. A method as claimed in claim 7 wherein the moiety comprises at least three carbons.

9. A method as claimed in claim 8 wherein the moiety comprises 4 carbons.

10. A method as claimed in claim 7 wherein the moiety comprises a terminal carbon having an amine group attached thereto.

11. A method as claimed in claim 7 wherein the thiopurine is a derivative of a compound selected from the group consisting of 6-thioguanine and 6-mercaptopurine.

12. A method as claimed in claim 7 wherein the thiopurine is selected from the group consisting of 6-(2-acetylvinylthio)guanine (AVTG) and 6-(2-acetylvinylthio)purine (AVTP).

13. A method as claimed in claim 7 wherein the tumor is selected from the group consisting of a blood-borne tumor and a solid-tumor.

14. A method as claimed in claim 13 wherein the blood-borne tumor is a leukemia.

15. A method as claimed in claim 13 wherein the solid tumor is selected from the group consisting of a kidney tumor, a colon tumor, an ovarian tumor and a skin tumor.

16. A method as claimed in claim 13 wherein the tumor upregulates glutathione.

17. A method as claimed in claim 16 wherein the tumor upregulates glutathione and is resistant to chemotherapy.

18. A method as claimed in claim 13 wherein the tumor has a high level of glutathione.
